(19) United States
(12) Patent Application Publication
(10) Pub. No.: US 2023/0140515 A1
(43) Pub. Date: May 4, 2023

(54) NITRIC OXIDE DONORS FOR USE IN SURGICAL RECOVERY

(71) Applicants: BAXTER INTERNATIONAL INC., Deerfield, IL (US); BAXTER HEALTHCARE SA, Glattpark (Opfikon) (CH)

(72) Inventor: John Devlin Foley, Libertyville, IL (US)

(21) Appl. No.: 17/979,095

(22) Filed: Nov. 2, 2022

Related U.S. Application Data
(60) Provisional application No. 63/275,176, filed on Nov. 3, 2021.

(51) Int. Cl. A61L 27/54; A61L 27/50

(52) U.S. Cl. CPC A61L 27/54; A61L 27/507; A61L 2300/204

(57) ABSTRACT

Disclosed herein are nitric oxide donors and methods of use thereof.

### FIELD

The present Specification relates to the use of nitric oxide donors to improve post-surgical outcomes.

### BACKGROUND

The wound healing cascade is a complex series of events involving four distinct phases: hemostasis, inflammation, proliferation, and remodeling. Nitric oxide (NO) plays a central role in the regulation of the vascular homeostasis and inflammation processes, as well as providing an antimicrobial effect.

Clinical evidence illustrates the impact of NO on the wound healing cascade; for example, states of NO deficiency including diabetes, malnutrition and steroid use are associated with delayed wound healing, providing clinical evidence of its importance. Further, supplementation with arginine, an NO precursor, improved wound healing in rat models. In addition, nitrate and nitrite (NO end products) are often present at elevated levels in wounds.

However, while the importance of NO in wound healing has been acknowledged, specific clinical applications are lacking. Further, NO’s high reactivity and short half-life make therapeutic design a challenge. Therefore, improved systems, devices, and methods are desirable.

### SUMMARY

The instant disclosure provides compositions and methods for accelerating revascularization and reducing seroma (a build-up of bodily fluids where tissue has been removed by surgery) formation in the post-surgical setting.

### DETAILED DESCRIPTION

While the importance of NO in wound healing has been described, the majority of the previous art has focused on cardiovascular applications, in particular for use in prevention of ischemia-reperfusion injury and neovascularization of ischemic diabetic limbs.

In contrast, the instant Specification discloses the use of NO donors (NOD) as an adjunct therapy for a variety of surgical techniques. In embodiments, NOD produce, or are converted to, NO, and promote a more rapid vascularization of the surgical site as well as reducing seroma in the surgical site.

### Definitions

“Administration,” or “to administer” means the step of giving (i.e. administering) a medical device, material or agent to a subject. The materials disclosed herein can be administered via a number of appropriate routes, but are typically employed in connection with a surgical procedure.

“Patient” means a human or non-human subject receiving medical or veterinary care.

“Pharmaceutical composition” means a formulation including an active ingredient such as an NOD. The word “formulation” means that there is at least one additional ingredient (such as, for example and not limited to, an albumin [such as a human serum albumin or a recombinant human albumin] and/or sodium chloride) in the pharmaceutical composition in addition to an active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free.

“Therapeutic formulation” means a formulation that can be used to treat and thereby alleviate a disorder or a disease and/or symptom associated thereof.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

Disclosed methods can be applied as stand-alone procedures, or in conjunction with current surgical procedures, for example staple reinforcement practices, or sealant uses. In embodiments, NOD are specifically not administered in cases involving surgical removal of cancerous tissue.

### Nitric Oxide Donor Compositions

Disclosed embodiments comprise the administration of NOD, for example to promote revascularization of a treatment site. NOD release NO by three general mechanisms (some donors utilize multiple pathways):
a. Spontaneous release by self-decomposition through thermal or photochemical means- S-nitrosothiols, diazeniumdiolates,oximines;
b. Release by chemical reactions with acid, alkali, metal and thiol- organic nitrites, nitrates and syndnonimines;
c. Release by enzymatic oxidation by nitric oxide synthases or oxidases- N-hydroxyguanidines.

Thus, in embodiments, NO is produced via self-decomposition, chemical reaction, or enzymatic oxidation.

NOD suitable for use in disclosed embodiments can comprise, for example, at least one of (±)-S-Nitroso-N-acetylpenicillamine (SNAP), S-Nitrosoglutathione (GSNO), streptozotocin, S-nitrosoglutathione, (+)-S-Nitroso-N-acetylepenicillamine, NOC-5, NOC-7, NOC-12, NOC-18, MAHMA NONOate, 3-morpholinosdnonimine, Angeli’s salt, NOR-1, NOR-2, NOR-3, NOR-4, NOR-5, DPTA NONOate, diethylamine NONOate, Spermine NONOate, sodium nitroprusside dehydrate, JS-K, Piloty’s acid, GEA 5583, PROLI NONOate, diethylamine NONOate/AM, fructose-SNAP-1, SIN-1A/gammaCD complex, BEC, nicorandil, 4-phenyl-3-furoxancarbonitrile, GEA 5024, GEA 3162, PAPA NONOate, glycol-SNAP-1, β-gal NONOate, 4-(p-methoxyphenyl)-1,3,2-oxathiazolylium-5-olate, molsidomine, hydroxyguanidine sulfate, tetrahydrobiopterin (THB) dihydrochloride, sulfo-NONate disodium salt, 10-nitrooleate, DD1, DD2, 4-chloro-4-phenyl-1,3,2-oxathiozolylium-5-olate, 4-phenyl-1,3,2-oxathiozolylium-5-olate, 4-trifluoro-4-phenyl-1,3,2-oxathiozolylium-5-olate, BNN3, 3-(methylnitrosamino)propionitrile, S-nitrosocapttopril, V-PYRRO/NO, SE 175, L-NMMA (citrate), NO-indomethacin, lansoprazole sulfone N-oxide, vinyl-L-NIO hydrochloride, AMI-1, DAF-FM DA, Ni-nitrosodiethylamine, N,N-dicarboxymethyl-N,N-dinotroso-p-phenylenediamine disodium salt, NO-aspirin 1, glycol-SNAP-2, 4AF-DA, (2S)-(+)-amino-6-iodoacetamidohexanoic acid, BEC ammonium salt, DAF-2 DA, DAN-1 EE hydrochloride, tricarbonyldichlororuthenium (II) dimer, guanylyl cyclase, hydroxyguanidine hemisulfate, N-cyclopropyl-Nprime- hydroxyguanidine hydrochloride, D,L-alpha-difluoromethylornithine hydrochloride, geranylgeranylacetone, N-ethyl-N-nitroso-1-propanamine-d4, N-acetyl-D,L-penicillamine disulfide, combinations thereof, and the like.

In embodiments, an NO stimulator can be used instead of, or in combination with, and NOD.

In embodiments, a nitrite salt can be utilized in disclosed compositions.

### NOD Compositions

Disclosed embodiments comprise NOD compositions in, for example, liquid, solid, or gel formulations. For example, in embodiments, disclosed liquid formulations for administration can further comprise, for example, a buffer, a salt, combinations thereof, and the like. Liquid formulations can be formulated as, for example, a spray, a gel, or the like.

Disclosed embodiments comprise NOD compositions in, for example, a solid or dry form, such as attached, bound, or mixed with a dry substrate. In embodiments comprising a solid or dry substrate, the substrate can comprise, for example, granules, for example granules comprising cross-linked hydrogels comprising at least one biologic or non-biologic polymer, for example proteins, polysaccharides, and synthetic polymers.

In embodiments the substrate polymer is biodegradable. Common biodegradable polymers include polylactic acid (PLA, also referred to as polylactide), polyglycolic acid (PGA), copolymers of PLA and PGA, polyamides, and copolymers of polyamides and polyesters.

In various embodiments, the substrate material comprises a recombinant polymer. In particular, the recombinant polymer can be a recombinant human collagen, such as, for example, recombinant human collagen type I, recombinant human collagen type III, or a combination thereof.

The biocompatible substrate material can also be based on a synthetic polymer. The synthetic absorbable polymer can be an aliphatic polyester polymer, an aliphatic polyester copolymer, or combinations thereof.

In embodiments, the polymer is capable of being cross-linked and hydrated to form a hydrogel. Exemplary polymers include proteins selected from gelatin, collagen (e.g. soluble collagen), albumin, hemoglobin, fibrinogen, fibrin, fibronectin, elastin, keratin, laminin, casein and derivatives and combinations thereof. Alternatively, the polymer may comprise a polysaccharide, such as a glycosaminoglycan (e.g., hyaluronic acid or chondroitin sulfate), a starch derivative, a cellulose derivative, a hemicellulose derivative, xylan, agarose, alginate, chitosan, and combinations thereof. As a further alternative, the polymer may comprise a non-biologic hydrogel-forming polymer, such as polyacrylates, polymethacrylates, polyacrylamides, polyvinyl polymers, polylactide-glycolides, polycaprolactones, polyoxyethylenes, and derivatives and combinations thereof.

A polysaccharide used as a biocompatible substrate material in disclosed embodiments can comprise, for example, cellulose, alkyl cellulose, methylcellulose, alkylhydroxyalkyl cellulose, hydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparan, heparan sulfate, dermatan sulfate, keratan sulfate, carrageenans, chitosan, starch, amylose, amylopectin, poly-N-glucosamine, polymannuronic acid, polyglucuronic acid, polyguluronic acid, derivatives of said polysaccharides, or combinations thereof.

Cross-linking of the polymer may be achieved in any conventional manner. For example, in the case of proteins, cross-linking may be achieved using a suitable cross-linking agent, such as an aldehyde, sodium periodate, epoxy compounds, and the like. Alternatively, cross-linking may be induced by exposure to radiation, such as γ-radiation or electron beam radiation. Polysaccharides and non-biologic polymers may also be cross-linked using suitable cross-linking agents and radiation. Additionally, non-biologic polymers may be synthesized as cross-linked polymers and copolymers. For example, reactions between mono- and poly-unsaturated monomers can result in synthetic polymers having controlled degrees of cross-linking. Typically, the polymer molecules will each have a molecular weight in the range from 20 kD to 200 kD, and will have at least one link to another polymer molecule in the network, often having from 1 to 5 links, where the actual level of cross-linking is selected in part to provide a desired rate of biodegradability in the ranges set forth below. Exemplary methods for producing molecular cross-linked gelatins are as follows.

Gelatin is obtained and placed in an aqueous buffer to form a non-cross-linked hydrogel, typically having a solids content from 1% to 70% w/w, usually from 3% to 10% by weight. The gelatin is then cross-linked, typically by exposure to either glutaraldehyde (e.g. 0.01% to 0.05% w/w, overnight at 0° C. to 15° C. in aqueous buffer), sodium periodate (e.g. 0.05 M, held at 0° C. to 15° C. for 48 hours) or 1-ethyl-3-(3-dimethylaminopropyl) carbodiimide (“EDC”) (e.g., 0.5% to 1.5% w/w, overnight at room temperature), or by exposure to about 0.3 to 3 megarads of gamma or electron beam radiation.

Alternatively, gelatin particles can be suspended in an alcohol, preferably methyl alcohol or ethyl alcohol, at a solids content of 1% to 70% by w/w, usually 3% to 10% by weight, and cross-linked by exposure to a cross-linking agent, typically glutaraldehyde (e.g., 0.01% to 0.1% w/w, overnight at room temperature). In the case of aldehydes, the pH can be held from about, for example, 6 to 11, preferably from 7 to 10. When cross-linking with glutaraldehyde, the cross-links are formed via Schiff bases which may be stabilized by subsequent reduction, e.g. by treatment with sodium borohydride. After cross-linking, the resulting granules may be washed in water and optionally rinsed in an alcohol, dried and resuspended to a desired degree of hydration in an aqueous medium having a desired buffer and pH. The resulting hydrogels may then be loaded into the applicators of the present invention, as described in more detail hereinafter. Alternatively, the hydrogels may be mechanically disrupted prior to or after cross-linking, also as described in more detail hereinafter. In embodiments, genipin can be employed as a cross-linker.

The extent of cross-linking of the polymer has an effect on several functional properties of the hydrogel including extrudability, adsorptiveness of surrounding biological fluids, cohesiveness, ability to fill space, swelling ability and ability to adhere to the tissue site. The extent of cross-linking of the polymeric hydrogel composition may be controlled by adjusting the concentration of cross-linking agent, controlling exposure to cross-linking radiation, changing the relative amounts of mono- and poly-unsaturated monomers, varying reaction conditions, and the like. Typically, the degree of cross-linking is controlled by adjusting the concentration of cross-linking agent.

The hydrogel compositions disclosed herein will typically have a solids content in the range from 1% by weight to 70% w/w. Optionally, the compositions may comprise at least one plasticizer as described in more detail below. Suitable plasticizers include polyethylene glycols, sorbitol, glycerol, and the like.

The equilibrium swell of the cross-linked polymers of the present disclosure may range from 400% to 5,000%, 400% to 3,000%, 400% to 2,000%, usually ranging from 400% to 1,300%, preferably being from 500% to 1100%, depending on its intended use. Such equilibrium swell may be controlled by varying the degree of cross-linking, which in turn is achieved by varying the cross-linking conditions, such as the type of cross-linking method, duration of exposure of a cross-linking agent, concentration of a cross-linking agent, cross-linking temperature, and the like.

Exposure to radiation, such as γ-radiation, may also be carried out in order to sterilize the compositions before or after packaging. When the compositions are composed of radiation-sensitive materials, it will be necessary to protect the compositions from the undesirable effects of sterilizing radiation. For example, in some cases, it will be desirable to add a stabilizer, such as ascorbic acid, in order to inhibit degradation and/or further excessive cross-linking of the materials by free radical mechanisms.

### Commercial Products / Kits

The present compositions and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (Irgacure 2959). Such irradiation is usually performed for an irradiation time of 1-60 min, but longer irradiation times may be applied, depending on the specific method. Sterile filtration can also be used for sterilization.

The material according to the present disclosure can be finally sterile-wrapped so as to retain sterility until use and packaged (e.g. by the addition of specific product information leaflets) into suitable containers (boxes, etc.).

According to further embodiments, the compositions can also be provided in kit form combined with other components necessary for administration of the material to the patient. For example, disclosed kits, such as for use in surgery, can further comprise, for example, a hemostatic material and at least one administration device, for example a buffer, a syringe, a tube, a catheter, forceps, scissors, gauze, a sterilizing pad or lotion.

The kits are designed in various forms based on the specific deficiencies they are designed to treat.

### Methods of Use

In embodiments, the NOD is administered by local administration to a treatment site. For example, in embodiments, the NOD can be administered in conjunction with a surgical procedure, such after the procedure, to aid in revascularization of the surgical site.

Further disclosed embodiments comprise administration of an NOD in conjunction with the use of a surgical sealant, for example a fibrin sealant, such as for adhering skin grafts, or tissue “flaps” associated with a wound.

### EXAMPLES

The following non-limiting Examples are provided for illustrative purposes only in order to facilitate a more complete understanding of representative embodiments. This example should not be construed to limit any of the embodiments described in the present Specification.

### Example 1

### Preparation of a Dry Powder NOD Formulation

Sodium nitrate is suspended in a solution, then spray dried to form a powder.

### Example 2

### Preparation of a Dry Powder NOD Formulation

Gelatin granules are suspended in a column of warm air in a fluidized bed approach. An NOD solution is sprayed onto the granules, forming granules of gelatin/NOD.

### Example 3

### Preparation of a Liquid NOD Formulation

An NOD is suspended in a solution of 0.9% (w/v) NaCl to form a liquid NOD formulation.

### Example 4

### NOD Use With a Surgical Procedure

A patient undergoes a hernia correction procedure utilizing a surgical mesh. Following implantation of the mesh, a dry powder NOD formulation is applied to the treatment site to accelerate revascularization.

### Example 5

### NOD Use With a Surgical Procedure

A patient undergoes a hernia correction procedure utilizing a surgical mesh. Following implantation of the mesh, a dry powder NOD formulation is applied to the treatment site to prevent seroma.

### Example 6

### NOD Use With a Surgical Procedure

A patient undergoes a surgical procedure utilizing staples to close the surgical incision. Prior to applying the staples and appropriate staple line reinforcement, a liquid NOD formulation is applied to the treatment site to accelerate revascularization.

### Example 6

### NOD Use With a Surgical Procedure

A patient undergoes a surgical procedure utilizing staples to close the surgical incision. Prior to applying the staples and appropriate staple line reinforcement, a liquid NOD formulation is applied to the treatment site to prevent seroma.

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method of accelerating post-surgical revascularization at a surgical treatment site, said method comprising administering a nitric oxide donor (NOD) to the site.

2. The method of claim 1, wherein said NOD comprises at least one of (±)-S-Nitroso-N-acetylpenicillamine (SNAP), S-Nitrosoglutathione (GSNO), streptozotocin, S-nitrosoglutathione, (+)-S-Nitroso-N-acetylepenicillamine, NOC-5, NOC-7, NOC-12, NOC-18, MAHMA NONOate, 3-morpholinosdnonimine, Angeli’s salt, NOR-1, NOR-2, NOR-3, NOR-4, NOR-5, DPTA NONOate, diethylamine NONOate, Spermine NONOate, sodium nitroprusside dehydrate, JS-K, Piloty’s acid, GEA 5583, PROLI NONOate, diethylamine NONOate/AM, fructose-SNAP-1, SIN-1A/gammaCD complex, BEC, nicorandil, 4-phenyl-3-furoxancarbonitrile, GEA 5024, GEA 3162, PAPA NONOate, glycol-SNAP-1, β-gal NONOate, 4-(p-methoxyphenyl)-1,3,2-oxathiazolylium-5-olate, molsidomine, hydroxyguanidine sulfate, tetrahydrobiopterin (THB) dihydrochloride, sulfo-NONate disodium salt, 10-nitrooleate, DD1, DD2, 4-chloro-4-phenyl-1,3,2-oxathiozolylium-5-olate, 4-phenyl-1,3,2-oxathiozolylium-5-olate, 4-trifluoro-4-phenyl-1,3,2-oxathiozolylium-5-olate, BNN3, 3-(methylnitrosamino)propionitrile, S-nitrosocapttopril, V-PYRRO/NO, SE 175, L-NMMA (citrate), NO-indomethacin, lansoprazole sulfone N-oxide, vinyl-L-NIO hydrochloride, AMI-1, DAF-FM DA, Ni-nitrosodiethylamine, N,N-dicarboxymethyl-N,N-dinotroso-p-phenylenediamine disodium salt, NO-aspirin 1, glycol-SNAP-2, 4AF-DA, (2S)-(+)-amino-6-iodoacetamidohexanoic acid, BEC ammonium salt, DAF-2 DA, DAN-1 EE hydrochloride, tricarbonyldichlororuthenium (II) dimer, guanylyl cyclase, hydroxyguanidine hemisulfate, N-cyclopropyl-Nprime- hydroxyguanidine hydrochloride, D,L-alpha-difluoromethylornithine hydrochloride, geranylgeranylacetone, N-ethyl-N-nitroso-1-propanamine-d4, and N-acetyl-D,L-penicillamine disulfide.

3. The method of claim 1, wherein said NOD comprises a nitrite salt.

4. The method of claim 1, wherein said NOD is administered as a dry powder.

5. The method of claim 1, wherein said NOD is administered as a liquid.

6. The method of claim 1, wherein said NOD is administered as a gel.

7. The method of claim 1, wherein said surgical treatment site does not comprise a site where cancerous tissue is removed.

8. The method of claim 1, wherein said surgical treatment site is further treated with staple line reinforcement.

9. A method of reducing post-surgical seroma formation at a surgical treatment site, said method comprising administering a nitric oxide donor (NOD) to the site.

10. The method of claim 9, wherein said NOD comprises at least one of (±)-S-Nitroso-N-acetylpenicillamine (SNAP), S-Nitrosoglutathione (GSNO), streptozotocin, S-nitrosoglutathione, (+)-S-Nitroso-N-acetylepenicillamine, NOC-5, NOC-7, NOC-12, NOC-18, MAHMA NONOate, 3-morpholinosdnonimine, Angeli’s salt, NOR-1, NOR-2, NOR-3, NOR-4, NOR-5, DPTA NONOate, diethylamine NONOate, Spermine NONOate, sodium nitroprusside dehydrate, JS-K, Piloty’s acid, GEA 5583, PROLI NONOate, diethylamine NONOate/AM, fructose-SNAP-1, SIN-1A/gammaCD complex, BEC, nicorandil, 4-phenyl-3-furoxancarbonitrile, GEA 5024, GEA 3162, PAPA NONOate, glycol-SNAP-1, β-gal NONOate, 4-(p-methoxyphenyl)-1,3,2-oxathiazolylium-5-olate, molsidomine, hydroxyguanidine sulfate, tetrahydrobiopterin (THB) dihydrochloride, sulfo-NONate disodium salt, 10-nitrooleate, DD1, DD2, 4-chloro-4-phenyl-1,3,2-oxathiozolylium-5-olate, 4-phenyl-1,3,2-oxathiozolylium-5-olate, 4-trifluoro-4-phenyl-1,3,2-oxathiozolylium-5-olate, BNN3, 3-(methylnitrosamino)propionitrile, S-nitrosocapttopril, V-PYRRO/NO, SE 175, L-NMMA (citrate), NO-indomethacin, lansoprazole sulfone N-oxide, vinyl-L-NIO hydrochloride, AMI-1, DAF-FM DA, Ni-nitrosodiethylamine, N,N-dicarboxymethyl-N,N-dinotroso-p-phenylenediamine disodium salt, NO-aspirin 1, glycol-SNAP-2, 4AF-DA, (2S)-(+)-amino-6-iodoacetamidohexanoic acid, BEC ammonium salt, DAF-2 DA, DAN-1 EE hydrochloride, tricarbonyldichlororuthenium (II) dimer, guanylyl cyclase, hydroxyguanidine hemisulfate, N-cyclopropyl-Nprime- hydroxyguanidine hydrochloride, DL-alpha-difluoromethylornithine hydrochloride, geranylgeranylacetone, N-ethyl-N-nitroso-1-propanamine-d4, and N-acetyl-D,L-penicillamine disulfide.

11. The method of claim 9, wherein said NOD comprises a nitrite salt.

12. The method of claim 9, wherein said NOD is administered as a dry powder.

13. The method of claim 9, wherein said NOD is administered as a liquid.

14. The method of claim 9, wherein said NOD is administered as a gel.

15. The method of claim 9, wherein said surgical treatment site does not comprise a site where cancerous tissue is removed.

16. The method of claim 9, wherein said surgical treatment site is further treated with staple line reinforcement.

17. A kit for use in performing a surgical procedure, comprising a nitric oxide donor (NOD) composition comprising at least one of (±)-S-Nitroso-N-acetylpenicillamine (SNAP), S-Nitrosoglutathione (GSNO), streptozotocin, S-nitrosoglutathione, (+)-S-Nitroso-N-acetylepenicillamine, NOC-5, NOC-7, NOC-12, NOC-18, MAHMA NONOate, 3-morpholinosdnonimine, Angeli’s salt, NOR-1, NOR-2, NOR-3, NOR-4, NOR-5, DPTA NONOate, β-gal NONOate, diethylamine NONOate, PAPA NONOate, sulfo-NONOate disodium salt, Spermine NONOate, sodium nitroprusside dehydrate, JS-K, Piloty’s acid, GEA 5583, PROLI NONOate, diethylamine NONOate/AM, fructose-SNAP-1, SIN-1A/gammaCD complex, BEC, nicorandil, 4-phenyl-3-furoxancarbonitrile, GEA 5024, GEA 3162, glycol-SNAP-1, 4-(p-methoxyphenyl)-1,3,2-oxathiazolylium-5-olate, molsidomine, hydroxyguanidine sulfate, tetrahydrobiopterin (THB) dihydrochloride, 10-nitrooleate, DD1, DD2, 4-chloro-4-phenyl-1,3,2-oxathiozolylium-5-olate, 4-phenyl-1,3,2-oxathiozolylium-5-olate, 4-trifluoro-4-phenyl-1,3,2-oxathiozolylium-5-olate, BNN3, 3-(methylnitrosamino)propionitrile, S-nitrosocapttopril, V-PYRRO/NO, SE 175, L-NMMA (citrate), NO-indomethacin, lansoprazole sulfone N-oxide, vinyl-L-NIO hydrochloride, AMI-1, DAF-FM DA, Ni-nitrosodiethylamine, N,N-dicarboxymethyl-N,N-dinotroso-p-phenylenediamine disodium salt, NO-aspirin 1, glycol-SNAP-2, 4AF-DA, (2S)-(+)-amino-6-iodoacetamidohexanoic acid, BEC ammonium salt, DAF-2 DA, DAN-1 EE hydrochloride, tricarbonyldichlororuthenium (II) dimer, guanylyl cyclase, hydroxyguanidine hemisulfate, N-cyclopropyl-Nprime-hydroxyguanidine hydrochloride, DL-alpha-difluoromethylornithine hydrochloride, geranylgeranylacetone, N-ethyl-N-nitroso-1-propanamine-d4, and N-acetyl-D,L-penicillamine disulfide.

18. The kit of claim 17, wherein said NOD composition comprises a dry powder.

19. The kit of claim 17, wherein said NOD composition comprises a liquid.

20. The kit of claim 17, wherein said NOD composition comprises a gel.
